(19) United States
(12) Patent Application Publication
(10) Pub. No.: US 2025/0017893 A1
(43) Pub. Date: Jan. 16, 2025

(54) FISETIN NANOPARTICLES

(71) Applicants: Luca Giuseppe Dalle Carbonare, Mezzane di Sotto (Verona) (IT); Maria Teresa Valenti, Mezzane di Sotto (Verona) (IT); Massimiliano Perduca, Castel D'Azzano (Verona) (IT)

(72) Inventors: Luca Giuseppe Dalle Carbonare, Mezzane di Sotto (Verona) (IT); Maria Teresa Valenti, Mezzane di Sotto (Verona) (IT); Massimiliano Perduca, Castel D'Azzano (Verona) (IT)

(21) Appl. No.: 18/713,198

(22) PCT Filed: Nov. 25, 2022
(86) PCT No.: PCT/IB2022/061431

§ 371 Date: May 24, 2024

(30) Foreign Application Priority Data
Nov. 26, 2021 (IT) 102021000029987

(51) Int. Cl. A61K 31/352; A61K 9/51; A61P 19/08

(52) U.S. Cl. CPC A61K 31/352; A61K 9/5123; A61K 9/5153; A61K 9/5192; A61P 19/08

(57) ABSTRACT

The present invention relates to a plurality of fisetin nanoparticles in poly(lactic-co-glycolic) acid (PLGA). The nanoparticles, characterized by a specific size, showed excellent stability and pharmacokinetics, therefore optimal properties for their use in pharmaceutical compositions or in the preparation of supplements and food supplements, to be used in particular for the prevention and treatment of bone diseases and for the treatment of cleidocranial dysplasia.

### FIELD OF THE INVENTION

The invention concerns fisetin nanoparticles in poly(lactic-co-glycolic) acid (PLGA) characterized by high stability and properties which make them optimal for oral administration in pharmaceutical and/or nutraceutical compositions.

### STATE OF THE ART

Flavonoids are plant pigments widely distributed in nature and regularly ingested in the diet. By virtue of their abundance in food products and their potential pharmacological and nutritional effects, flavonoids have attracted considerable interest in both nutraceutical and drug development.

It has been demonstrated in the literature that many flavonoids produce biological effects through different mechanisms of action. For example, some in vitro experiments have shown that flavonoids are able to modulate osteogenesis by influencing the physiology of bone-forming cells, i.e. osteoblasts [Trzeciakiewicz, A., V. Habauzit, and M.-N. J. N. R. R. Horcajada, When nutrition interacts with osteoblast function: molecular mechanisms of polyphenols. 2009. 22 (1): pp. 68-81].

Among flavonoids, fisetin, which is found in fruits and vegetables such as strawberries, persimmons, apples, grapes, onions and cucumbers, is particularly studied because of its properties

Fisetin (3,3′,4′,7-tetrahydroxyflavone) is a polyphenol of formula:

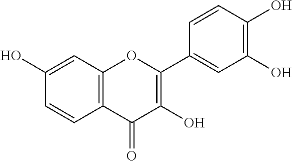 [SMILES: O=c1c(O)c(-c2ccc(O)c(O)c2)oc2cc(O)ccc12]

In vitro experiments have shown that fisetin can induce apoptosis in lung and prostate cancer cells [Sung, B., M. K. Pandey, and B. B. J. M. p. Aggarwal, Fisetin, an inhibitor of cyclin-dependent kinase 6, down-regulates nuclear factor-κB-regulated cell proliferation, antiapoptotic and metastatic gene products through the suppression of TAK-1 and receptor-interacting protein-regulated IκBα kinase activation. 2007. 71 (6): pp. 1703-1714, e Khan, N., et al., Fisetin, a novel dietary flavonoid, causes apoptosis and cell cycle arrest in human prostate cancer LNCaP cells. 2008. 29 (5): pp. 1049-1056] and that it exerts antioxidant properties in retinal epithelial and neuronal cells [Hanneken, A., et al., Flavonoids protect human retinal pigment epithelial cells from oxidative-stress-induced death. 2006. 47 (7): pp. 3164-3177, and Ishige, K., et al., Flavonoids protect neuronal cells from oxidative stress by three distinct mechanisms. 2001. 30 (4): pp. 433-446] as well as anti-inflammatory effects in macrophages and fibroblasts [Liu, S.-H., et al., Fisetin inhibits lipopolysaccharide-induced macrophage activation and dendritic cell maturation. 2010. 58 (20): pp. 10831-10839, and Funakoshi-Tago, M., et al., Anti-inflammatory activity of structurally related flavonoids, Apigenin, Luteolin and Fisetin. 2011. 11 (9): pp. 1150-1159]. By testing fisetin in in vivo models, it has been shown that it can counteract tumor growth [Khan, N., et al., A novel dietary flavonoid fisetin inhibits androgen receptor signaling and tumor growth in athymic nude mice. 2008. 68 (20): pp. 8555-8563], osteoarthritis [Zheng, W., et al., Fisetin inhibits IL-1β-induced inflammatory response in human osteoarthritis chondrocytes through activating SIRT1 and attenuates the progression of osteoarthritis in mice. 2017. 45: pp. 135-147] and rheumatoid arthritis [Léotoing, L., et al., The polyphenol fisetin protects bone by repressing NF-κB and MKP-1-dependent signaling pathways in osteoclasts. 2013. 8 (7): pp. e68388].

Furthermore, fisetin has been shown to prevent inflammation-induced bone loss and promote Runx2 upregulation in order to stimulate primary osteoblastic activity, thus enhancing the mineralization process [Léotoing, L., et al., The flavonoid fisetin promotes osteoblasts differentiation through Runx2 transcriptional activity. 2014. 58 (6): pp. 1239-1248].

Despite fisetin beneficial effects in the prevention and treatment of various diseases, its low solubility in water, chemical instability, poor absorption, and rapid metabolism dramatically contribute to its low oral bioavailability, thus severely limiting its clinical application.

In this context, nanoencapsulation is one of the pharmaceutical drug delivery techniques that can be used to try to overcome these physicochemical and pharmacokinetic limitations.

Some attempts of fisetin nanoencapsulation are known in the literature.

For example, in an article by N. Mignet [N. Mignet, J. Seguin, M. Ramos Romano, L. Brullé, Y. S. Touil, D. Scherman, M. Bessodes, G. G. Chabot, Development of a liposomal formulation of the natural flavonoid fisetin, Int. J. Pharm. 423 (2012) pp. 69-76] a formulation obtained by encapsulation of a nanoemulsion of fisetin in liposomes is described, which proved to be particularly bioavailable and effective as an antitumor agent in Lewis lung carcinoma.

In another case, fisetin encapsulation in monomethyl-poly(ethylene glycol)-poly(ε-caprolactone) copolymer micelles improved the therapeutic effect in colon cancer [Y. Chen, Q. Wu, L. Song, T. He, Y. Li, L. Li, W. Su, L. Liu, Z. Qian, C. Gong, Polymericmicellesencapsulating fisetin improve the therapeutic effect in colon cancer, ACS Appl. Mater. Interfaces 7 (2015) 534-542].

Still, in an article by M. Sechi [M. Sechi, D. N. Syed, N. Pala, A. Mariani, S. Marceddu, A. Brunetti, H. Mukhtar, V. Sanna, Nanoencapsulation of dietary flavonoid fisetin: Formulation and in vitro antioxidant and α-glucosidase inhibition activities, Materials Science and Engineering C68 (2016), pp. 594-602]fisetin, which was formulated in polymeric nanoparticles based on poly(ε-caprolactone) (PCL) and poly(D,L-lactic acid-co-glycolic acid)-block-(ethylene glycol)-carboxylic acid (i.e. PLGA-PEG-COOH), suitable for oral controlled release, was effective in postprandial glycemic control.

In A. Kadari [Kadari, A., et al., Enhanced oral bioavailability and anticancer efficacy of fisetin by encapsulating as inclusion complex with HPβCD in polymeric nanoparticles, Drug Delivery (2017), 24 (1): pp. 224-232] fisetin was encapsulated in PLGA nanoparticles in the form of a complex with HPβCD (hydroxy-propyl-β-cyclodextrin). With reference to the latter preparation, it is important to note that fisetin, being in a complex form with other molecules, is not released by the polymeric matrix in a free and immediately bioavailable form, but rather in a bound form, inside the fisetin-excipient complex, and therefore it is less active.

Furthermore, in terms of preparation, both articles by Sechi and Kadari describe a preparation of nanoparticles through nanoprecipitation and double emulsion methods, i.e. a rather long and delicate procedure overall.

In particular, in Kadari's work, fisetin is first complexed with hydroxypropyl-β-cyclodextrin to increase its solubility in water, and then emulsified to obtain PLGA nanoparticles. In Sechi's article, however, the authors used a nanoprecipitation and two different polymers (PLGA-peg and polycapryl lactone).

Other studies on nanoencapsulation of fisetin in PLGA are described in the very recent article by Liu [Liu, W.-Y., et al., Nanoformulation Development to Improve the Biopharmaceutical Properties of Fisetin Using Design of Experiment Approach., Molecules, 2021. 26 (10): pp. 3031] in which, through drug design techniques (DOE-design of experiment) the authors search the conditions for optimizing the preparation of fisetin nanoparticles according to the amount of PLGA and PVA used in the synthetic process. According to the results of their experiments, the best preparation conditions were obtained when 5 mg of fisetin, PVA in a 0.5% (w/v) concentration and 75.3 mg of PLGA were used, thus obtaining nanoparticles of fisetin-PLGA with a diameter size equal to 187.9 nm. As is evident, the topic of obtaining formulations of fisetin nanoparticles is still an open topic and not yet fully explored.

The possibility of making a stable and bioavailable fisetin composition for oral administration is therefore a need still felt in the clinical field, in order to have an effective formulation that can be administered in various fields and for various applications, from pharmaceutical to dietary-nutritional ones.

The object of the present invention is therefore to make available a new fisetin formulation which is capable of providing effective answers to the needs and issues described above.

### SUMMARY OF THE INVENTION

The inventors surprisingly found that nanoparticles of fisetin in PLGA having diameters in the range from 120 to 160 nm were particularly stable.

In a main embodiment, the invention therefore relates to a plurality of fisetin nanoparticles in poly(lactic-co-glycolic) acid (PLGA), wherein each particle has a diameter in the range from 120 to 160 nm.

Particle sizes were obtained by DLS (Dynamic light scattering) technique using Nano Zeta Sizer ZS, ZEN3600 from Malvern Instrument.

The diameter sizes of said nanoparticles are preferably in the range from 130 to 150 nm, more preferably in the range from about 135 to about 143 nm. Preferably said plurality of particles have a polydispersity index PDI in the range from 0.09 to 0.13.

In a further aspect, the invention concerns a method of preparing a plurality of fisetin nanoparticles in poly(lactic-co-glycolic) acid (PLGA), wherein each particle has a diameter in the range from 120 to 160 nm, comprising the following steps:
(a) dissolving poly(lactic-co-glycolic) acid (PLGA) and fisetin, in a weight ratio in the range from 15:1 to 20:1, in DMSO, until a clear and homogeneous organic phase is obtained;
(b) adding dropwise and under stirring said organic phase obtained in step (a) to an aqueous solution of polyvinyl alcohol (PVA) and then allowing the organic phase to evaporate slowly, thus obtaining an aqueous suspension;
(c) centrifuging the aqueous suspension of step (b) and eliminating the supernatant in order to obtain the plurality of fisetin nanoparticles in PLGA.

The inventors, in fact, surprisingly discovered that the adoption of these specific weight ratios between fisetin and poly(lactic-co-glycolic) acid (PLGA), together with the use of DMSO as an organic solvent, allowed to obtain the plurality of fisetin nanoparticles in PLGA in a totally reproducible manner and in the desired size.

Said plurality of nanoparticles could also be stored and used in the form of an aqueous suspension.

In an alternative embodiment of the invention, therefore, the invention also concerns an aqueous suspension of said plurality of fisetin nanoparticles in PLGA, wherein the aqueous solution is phosphate buffered saline (PBS) at pH 7.4 or an 0.9% (w/w) aqueous solution of sodium chloride.

The invention also concerns a composition comprising the plurality of nanoparticles of the invention, or the aqueous suspension of the plurality of particles of the invention, and excipients.

In an advantageous embodiment of the invention, the composition of the invention is a nutraceutical composition comprising the plurality of particles of the invention, or the aqueous suspension of the plurality of particles, and nutraceutically acceptable excipients.

In a further advantageous embodiment of the invention, the composition of the invention is a food composition comprising the plurality of nanoparticles according to the invention, or the aqueous suspension of the plurality of particles, and at least one excipient suitable for food use. In fact, the properties of the fisetin nanoparticles in PLGA make the composition of the invention useful also for administration as a food supplement for skeletal system improvement.

In another aspect, as will be evident from the experimental part, the invention concerns a pharmaceutical composition comprising the plurality of particles of the invention, or the aqueous suspension of the plurality of particles, and at least a pharmaceutically acceptable excipient.

Advantageously, the composition of the invention, wherein each particle has a diameter in the range from 120 to 160 nm, is able to concentrate the action of the active ingredient through a single bioavailability peak, thus allowing to administer lower drug concentrations.

The inventors also discovered that said compositions exhibited a bioavailability profile suitable for oral administration and were particularly effective in the prevention and/or treatment of certain bone diseases.

Therefore, in a further aspect, the invention concerns the pharmaceutical composition of the invention for use in the prevention and/or treatment of bone diseases, in particular osteoporosis, and for the treatment of cleidocranial dysplasia.

As will be evident, in fact, from the results shown in the following experimental part, the formulation of nanoparticles according to the invention proved capable of stimulating osteogenic differentiation and osteoblastic maturation.

Finally, under a further aspect, the invention concerns the pharmaceutical or nutraceutical composition of the invention for use, in the veterinary field, in the prevention and/or treatment of bone diseases in animals.

The nutraceutical composition of the invention has proved to be particularly advantageous thanks to the skeletal system improvement achieved through its use.

Thus, in another aspect, the invention concerns a nutraceutical composition according to the invention for use in skeletal system improvement, wherein said skeletal system improvement is selected from the group consisting of stimulation of osteogenic cell differentiation, restoration of osteogenic maturation and increase in osteoblast production activity.

### DETAILED DESCRIPTION OF THE INVENTION

The invention therefore concerns a plurality of fisetin nanoparticles in poly(lactic-co-glycolic) acid (PLGA), wherein each particle has a diameter in the range from 120 to 160 nm.

Particle sizes were measured by DLS (Dynamic light scattering) technique using the Nano Zeta Sizer ZS, ZEN3600 from Malvern Instrument.

Diameter size of said nanoparticles are preferably in the range from 130 to 150 nm, more preferably in the range from about 135 to about 143 nm.

Preferably, said plurality of particles has a polydispersity index PDI in the range from 0.09 to 0.13.

When the term “PLGA” is used in the present invention, it means poly(lactic-co-glycolic acid) polymer.

In a further aspect, the invention concerns a method of preparing said plurality of fisetin nanoparticles in poly(lactic-co-glycolic) acid (PLGA), wherein each particle has a diameter in the range from 120 to 160 nm, comprising the following steps:
(a) dissolving PLGA and fisetin, in a weight ratio in the range from 15:1 to 20:1, in DMSO, until a clear and homogeneous organic phase is obtained;
(b) adding dropwise and under stirring said organic phase obtained in step (a) to an aqueous solution of polyvinyl alcohol (PVA) and then allowing the organic phase to evaporate slowly, thus obtaining an aqueous suspension;
(c) centrifuging the aqueous suspension of step (b) and eliminating the supernatant in order to obtain the plurality of fisetin nanoparticles in PLGA.

The inventors, in fact, surprisingly discovered that the adoption of these specific weight ratios between fisetin and poly(lactic-co-glycolic) acid (PLGA), together with the use of DMSO as an organic solvent, allowed to obtain the plurality of fisetin nanoparticles in PLGA in a totally reproducible manner and in the desired size.

Preferably, the dimethyl sulfoxide (DMSO) of step (a) is 100% (w/w) DMSO. Preferably, the polyvinyl alcohol (PVA) used in step (b) is an aqueous solution of PVA in a concentration from 0.3 to 1%, more preferably of about 0.5%. Preferably, the centrifugation of step (c) is carried out at a temperature in the range from 0 to 10° C., more preferably at about 4° C.

Preferably, said centrifugation is carried out at a rate in the range from 3200 to 29000 rpm, more preferably at about 15500 rpm.

Still preferably, said centrifugation is carried out for a time between 15 and 30 minutes, more preferably for about 20 minutes.

The preparation method according to the invention may also provide, after step (c) of obtaining the plurality of fisetin nanoparticles in PLGA, for the subsequent rinsing of the same with ultrapure water.

In another aspect, the invention concerns a plurality of particles obtainable by the method of the invention, wherein each particle has a diameter in the range from 120 to 160 nm and wherein said plurality of particles has a polydispersity index PDI in the range from 0.09 to 0.13.

In an advantageous embodiment of the invention, the plurality of fisetin nanoparticles in PLGA, wherein each particle has a diameter in the range from 120 to 160 nm, according to the invention can be stored and preserved for long periods at the temperature of 4° C. in the form of aqueous suspension, if resuspended in phosphate buffered saline (PBS) at pH 7.4, or in a sodium chloride solution at a concentration of about 0.9% (w/w).

In an alternative embodiment of the invention, therefore, the invention also concerns an aqueous suspension of the plurality of fisetin nanoparticles in PLGA or the fisetin nanoparticles in PLGA obtainable by the method of the invention, wherein the aqueous solution is phosphate buffer saline (PBS) at pH 7.4 or sodium chloride at a concentration of about 0.9% (w/w).

The invention also concerns a composition comprising the plurality of nanoparticles of the invention, or the aqueous suspension of the plurality of nanoparticles of the invention, and excipients.

This composition proved to be suitable to be used in the preparation of drugs, supplements, or food supplements.

In fact, the experiments demonstrated a bioavailability and stability profile of the compositions of fisetin in PLGA such as to be able to use them effectively for making said products.

In a further advantageous aspect, the invention therefore concerns a nutraceutical composition comprising the plurality of particles of the invention, or the aqueous suspension of the plurality of nanoparticles of the invention, and at least a nutraceutically acceptable excipient.

In another aspect, the invention therefore also relates to a pharmaceutical composition comprising the plurality of particles of the invention, or the aqueous suspension of the plurality of nanoparticles of the invention, and at least a pharmaceutically acceptable excipient.

When, moreover, the plurality of nanoparticles of the invention preferably had a PDI in the range from 0.09 to 0.13, as will be evident from the following experimental part, the pharmaceutical composition of the invention advantageously had, in terms of biological profile, a more predictable and more effective response upon interaction with the drug, which avoided the dispersion of its action at different timepoints by virtue of the limited dimensional dispersion of its particles. The inventors therefore believe that, when the pharmaceutical composition of the invention has said polydispersity index PDI, it is more capable of concentrating the action of the active ingredient, through a single bioavailability peak, thus allowing to administer even lower concentrations of drug.

Preferably the composition of the invention comprises a unit dosage of fisetin in the range from 50 to 110 mg, more preferably from 70 mg to 90 mg.

Preferably, when said composition of the invention is a pharmaceutical or nutraceutical composition, said unit dosage of fisetin is administered once a day. Preferably, the composition of the invention is for oral use, preferably in the form of granules, powder, syrup, tablet, capsule, gel, aqueous suspension and liquid.

In a preferred embodiment said composition is in powder form.

In a further preferred embodiment said composition is in the form of a granulate.

In a further preferred embodiment said composition is in the form of an aqueous suspension.

Advantageously, when the composition is in the form of a powder, granulate or suspension, it is in a sachet format, preferably in a single-dose sachet format for oral administration.

Said composition for oral use can also be immediate or modified release according to the specific excipients selected for the its formulation.

In yet another aspect, the invention concerns the pharmaceutical composition according to the invention for use in the prevention and/or treatment of bone diseases, such as for example osteoporosis.

In fact, as will be evident from the results reported in the following experimental part, the pharmaceutical composition according to the invention proved capable of stimulating osteoblastic differentiation.

This ability makes the pharmaceutical composition of the invention particularly suitable for use in the treatment of all diseases typically characterized by a deficit of osteoblastic activity, such as for example hemoglobinopathies (such as sickle cell anemia and thalassemia) or endocrinopathies (such as acromegaly and hypothyroidism).

For the same reason, the composition of the invention may be particularly useful in clinical situations characterized by a picture of unmarked bone demineralization, or osteopenia, therefore not yet susceptible to pharmacological treatment. In another aspect, therefore, the invention concerns the use of the composition in the prevention and/or treatment of osteopenia.

The composition of the invention also proves to be particularly useful even for use after completion of a specific therapy for the primary or secondary prevention of skeletal fractures, such as for example in cases of interruption of treatment with antiresorptive or anabolic steroids, in which it could effectively be associated to vitamin D administration, or again in association with common antiresorptive therapies to promote osteoblastic activity and optimize the treatment. In another aspect, therefore, the invention concerns the use of the composition in the prevention and/or treatment of a bone disease, wherein the composition is associated with vitamin D.

Likewise, the composition of the invention is indicated in cases where acute or chronic treatment with glucocorticoids is required to prevent the negative effect of the same treatment on osteoblast activity.

In addition, fisetin also proved capable of stimulating the maturation of osteoblastic cells, as will be clear from the following experimental part, in the case of cells affected by cleidocranial dysplasia.

Thus, in yet a further aspect, the invention relates to a pharmaceutical composition comprising a plurality of PLGA fisetin nanoparticles, each having a diameter in the range of 120 to 160 nm, for use in the prevention of cleidocranial dysplasia.

Furthermore, the nutraceutical composition of the invention has proved to be particularly advantageous thanks to the skeletal system improvement achieved through its use.

Therefore, in another aspect, the invention concerns a nutraceutical composition according to the invention for use in skeletal system improvement, wherein said skeletal system improvement is selected from the group consisting of stimulation of osteogenic cell differentiation, restoration of osteogenic maturation and increase in osteoblast production activity.

In a further aspect, the invention concerns the pharmaceutical or nutraceutical composition of the invention for use, in the veterinary field, in the prevention and/or treatment of bone diseases in animals.

Finally, given the properties exhibited by the fisetin nanoparticles in PLGA according to the invention, in a further aspect, the invention therefore also relates to a food composition comprising the plurality of particles of the invention, or the aqueous suspension of the plurality of nanoparticles of the invention, and at least a food-acceptable excipient.

In conclusion, the inventors have surprisingly demonstrated that the fisetin nanoparticles in PLGA according to the invention are endowed with particular stability and excellent pharmacokinetics, therefore they can effectively be used to promote osteogenesis, and are therefore particularly suitable for the prevention and treatment of bone diseases, such as osteoporosis, in particular, as well as in the treatment of cleidocranial dysplasia.

### EXPERIMENTAL PART

### Example 1—Preparation of Fisetin Nanoparticles in PLGA

### Materials

PLGA (poly [DL-lactide-co-glycolide], lactide-glycolide 50:50 ratio, CAS 26780-50-7), PVA (poly [vinyl alcohol], CAS 9002-89-5), acetone (purity ≥99% 1.00013) purchased from Merck, dimethyl sulfoxide (DMSO, purity ≥99% D-5879) purchased from Sigma-Aldrich. Fisetin purchased from Santa Cruz Biotechnology (SC-276440).

### Preparation

The protocol used for preparing fisetin-loaded PLGA nanoparticles (PLGA [Fis]) is based on a single emulsion evaporation method, under sterile conditions at 20° C.

20 mg of PLGA polymer and 4 mM fisetin (1.14 mg) were dissolved together in 1 mL of 100% DMSO; the organic phase obtained was then added dropwise under stirring (2000 RPM) to 10 mL of a 0.5% (w/w) polyvinyl alcohol (PVA) aqueous solution and left overnight to evaporate in order to eliminate the organic phase. Subsequently, the aqueous suspension was subjected to centrifugation at 4° C. and 15500 g for 20 min (using an Eppendorf centrifuge, 5804R model) and the nanoparticles were collected and washed twice with 10 mL of Milli-Q water.

Finally, the purified nanoparticles were resuspended in 1 mL of phosphate buffered saline (PBS) at pH 7.4 for subsequent analysis and storage at 4° C.

### Example 2—Characterization of Fisetin Nanoparticles in PLGA

Determination of nanoparticle size and zeta potential (ζ)

The size and zeta potential (ζ) of the PLGA nanoparticles were determined at 25° C. by DLS (Dynamic light scattering) technique using Nano Zeta Sizer ZS instrument, ZEN3600 from Malvern Instruments.

For determining the nanoparticles size, the nanoparticles suspended in PBS prepared according to Example 1, were diluted by 20-fold in PBS; while for determining the zeta potential (ζ) they were placed in a 10 mM NaClO4 solution at pH 7.5; the data were collected in triplicate and analyzed with the ZetaSizer software.

The following values were obtained:

Particle Diameter (nm): 139.2 ± 4.0
PDI: 0.11 ± 0.02
Zeta Potential ζ (mV): −10.20 ± 0.30

As is evident, the fisetin nanoparticles in PLGA prepared according to the process of the invention also had a significantly low PDI (Poly Dispersity Index), indicating the optimal narrow dimensional distribution of the particle diameters around their mean value.

The PDI is, in fact, defined as the standard deviation (o) of the particle diameter distribution divided by the mean particle diameter. This index is used to estimate the average uniformity of a particle sample and lower PDI values correspond to a smaller size distribution in the particle sample.

In the specific case of nanoparticles intended for use in pharmaceutical compositions, having a low DPI is particularly important because this allows for a more predictable response to drug interaction in terms of biological profile (since its bioavailability is also linked to its rapidity of dissolution, which is in turn proportional to its surface area and therefore to the particle size), and more effective, avoiding a dispersion of its action at different timepoints due to its particle size dispersion, and therefore being able to concentrate its action in a single bioavailability peak, thus allowing to administer lower drug concentrations.

### Spectroscopy, Encapsulation Efficiency (EE) and Drug Loading (DL)

Absorption and emission spectroscopy was used to confirm the presence of fisetin within the nanoparticles.

The absorbance of a sample of nanoparticles resuspended in PBS obtained according to Example 1 was analyzed using a Thermo Fisher Evolution 201 UV-Visible spectrophotometer, in the range between 250 and 600 nm. The emission profile of the sample at 360 nm, i.e. at the fisetin excitation wavelength, was also recorded using a Jasco FP-8200 spectrofluorometer.

Both determinations confirmed the presence of fisetin in the particles.

Therefore, the encapsulation efficiency (EE) and the drug loading (Drug Loading, DL) were calculated.

To quantify the amount of trapped fisetin, the fisetin nanoparticles were dissolved in DMSO and analyzed using a calibration line. Encapsulation efficiency (EE) and Drug Loading (DL) were estimated using the following equations:

$$EE ( \% ) = \frac{{Fisetin}_{loaded}}{{Fisetin}_{fed}} \times 100 \\ DL ( \% ) = \frac{mg di {Fisetin}_{loaded}}{mg di PLGA} \times 100$$

In particular, therefore, the EE (%) value is determined as the percentage ratio between the amount of fisetin disappeared from the solution because of trapping in the PLGA nanoparticle and the amount of fisetin initially available, while the DL (%) value is determined as the percentage ratio between the amount of fisetin trapped in the PLGA nanoparticles and the amount of PLGA present in the nanoparticles and it is an index of the polymer's ability to retain the drug.

The higher the DL (%) value, and therefore the more drug the polymer unit is able to retain, the more efficient the method of preparing the nanoparticle, which allows for the delivery of more drug with smaller amounts of the same.

Said determinations carried out with the nanoparticles according to the invention, provided the following values:

EE(%): 75.57 ± 4.21
DL(%): 23.51 ± 5.07

As is evident, the fisetin nanoparticles in PLGA prepared according to the invention exhibited significantly high values of EE (%) and DL (%).

### Example 3—Comparative Tests for the Properties of Fisetin Nanoparticles in PLGA According to the Invention and Fisetin Nanoparticles of the Prior Art

The characteristics of the fisetin nanoparticles in PLGA prepared according to the invention (indicated with PLGA-FIS) were compared with the characteristics of the nanoparticles prepared according to the prior art, in particular according to the procedures reported in the description of the prior art in the article by Kadari et Al (indicated with KAD), in the article by Sechi et Al (indicated with F1, F2 and F3) and in the article by Liu et Al (indicated with LIU).

The following Table 1 shows the comparison between the nanoparticles according to the invention, PLGA-FIS, and the nanoparticles of Kadari et Al (KAD) with relation to the most significant parameters:

TABLE 1
Comparison between characteristics of fisetin nanoparticles
obtained according to the invention (PLGA-FIS) and those
obtained according to Kadari et Al (KAD).

| | Particle diameter size | | Zeta potential ζ |
|---|---|---|---|
| Nanoparticles | (nm) | PDI | (mV) |
| PLGA-FIS | 139.2 ± 4.0 | 0.11 ± 0.02 | −10.20 ± 0.30 |
| KAD | 87.3 ± 10.0 | 0.25 ± 0.01 | −8.71 ± 0.03 |

As is evident from the data in Table 1, in the case of fisetin nanoparticles prepared according to Kadari's article, nanoparticles of smaller size but decidedly more polydisperse were obtained. The PDI value is even more than double the PDI value of the nanoparticles according to the invention. Among other things, the various colloids also exhibited a lower stability in suspension as is evident from the less negative value of the zeta potential.

In addition, the nanoparticles prepared by Kadari released fisetin complexed with cyclodextrin, therefore a fisetin decidedly less bioavailable compared to the free fisetin that would be released by the PLGA nanoparticle according to the invention.

Therefore, the comparison of the characteristics of fisetin nanoparticles according to the invention with those prepared according to the method by Kadari et Al demonstrated the clear superiority and greater efficacy in terms of fisetin bioavailability, of the fisetin nanoparticles in PLGA according to the invention.

The following Table 2 shows instead the comparison between the most significant parameters of the nanoparticles prepared according to the invention (PLGA-FIS) and the nanoparticles by Sechi et Al, which were prepared in three different ways (F1, F2 and F3), depending on the different amount of the two polymers PCL and PLGA-PEG-COOH used, as indicated in the same publication.

TABLE 2
Comparison between the characteristics of fisetin nanoparticles
obtained according to the invention (PLGA-FIS) and those
obtained according to Sechi et Al (F1, F2 and F3).

| | Particle diameter size | | |
|---|---|---|---|
| Nanoparticles | (nm) | PDI | DL (%) |
| PLGA-FIS | 139.2 ± 4.0 | 0.11 ± 0.02 | 23.51 ± 5.07 |
| F1 | 146.2 ± 2.3 | 0.12 ± 0.05 | 4.10 ± 0.20 |
| F2 | 198.7 ± 6.0 | 0.16 ± 0.02 | 3.74 ± 0.10 |
| F3 | 165.4 ± 3.3 | 0.15 ± 0.02 | 3.49 ± 0.10 |

As is evident from the data in Table 2, in the case of the fisetin nanoparticles prepared according to the invention it was possible to obtain nanoparticles with a smaller size and an even better PDI index.

Furthermore, a drug load value (DL) was obtained, which was even from about 6 to about 7 times higher than that obtained according to the methods by Sechi et Al.

This huge difference is reflected in an extraordinary efficacy of the fisetin nanoparticles in PLGA according to the invention in being able to carry large amounts of drug, even at low doses.

This characteristic is clearly highly desired in a drug because it also proportionally reduces the potential adverse effects associated with the administration of high amounts of nanoparticles, as well as improving the stability profile of the formulations over time.

Therefore, the comparison of the characteristics of the fisetin nanoparticles according to the invention with those prepared according to the method by Sechi et Al demonstrated the clear superiority of the fisetin nanoparticles in PLGA according to the invention.

Finally, the following Table 3 shows the comparison between the nanoparticles according to the invention PLGA-FIS and the nanoparticles by Liu et Al (LIU) with relation to the most significant parameters:

TABLE 3
Comparison between the characteristics of fisetin nanoparticles
obtained according to the invention (PLGA-FIS) and
those obtained according to Liu et Al (LIU).

| | Particle diameter size | | Zeta potential ζ |
|---|---|---|---|
| Nanoparticles | (nm) | PDI | (mV) |
| PLGA-FIS | 139.2 ± 4.0 | 0.11 ± 0.02 | −10.20 ± 0.30 |
| LIU | 187.9 ± 6.1 | 0.12 ± 0.01 | −29.20 ± 1.60 |

As is evident from the data in Table 3, in the case of fisetin nanoparticles prepared according to Liu's article, very stable nanoparticles were obtained, as evidenced by the really good zeta potential value, having a low polydispersity index, as evidenced by the comparable PDI value, but still very high particle sizes, about 35% higher than those obtainable with the method according to the invention.

Given the well-known importance of being able to operate with particles having the smallest possible size, the particles according to the invention represent the optimal nanoparticles for the purposes of preparing a pharmaceutical composition, also when compared to the nanoparticles prepared according to Liu's method.

Finally, it is observed that the particles of the invention also exhibited an extraordinarily high value, with respect to their size, of encapsulation efficiency EE % (over 75%, as reported in Example 2); completely unusual and unexpected data according to the literature data, in particular according to the data described by LIU.

Ultimately, the comparative tests showed that the fisetin nanoparticles in PLGA according to the invention, having an intermediate size compared to the size of all the nanoparticles prepared in the prior art, surprisingly exhibited an absolutely unpredictable behaviour, with respect to the different observed properties, highlighting a PDI index better than that of any other nanoparticle of the prior art, both dimensionally smaller and larger, an adequate zeta potential to ensure their stability, and an exceptional encapsulation efficiency and drug loading capacity compared to known nanoparticles.

### Example 4—Intestinal Epithelial Tissue Crossing Test

### 3D Fluid Dynamic Intestinal Model for Simulating a Systemic Administration of Fisetin Nanoparticles in PLGA.

A compartmental fluidic device, marketed under the name MIVO by the company React4life S.r.l., was used to perform this in vitro drug efficacy test.

The 3D fluidic model, which was supposed to demonstrate the ability of the fisetin nanoparticles in PLGA of the invention to cross the intestinal barrier, was performed using the following three-step method:
1) 24 wells containing human intestinal tissue (EpiIntestinal by Mattek) were placed and cultured inside the device, forming two fluidically independent chambers: the donor and the receiver;
2) both chambers were then filled with culture medium;
3) the receiver chamber was connected to the peristaltic pump to form a closed-loop fluidic circuit containing 3.8 mL of medium circulating at a rate of 0.3 cm/s, to simulate the capillary flowrate. Subsequently, a medium containing the fisetin nanoparticles in PLGA of the invention was added to the donor chamber.

The amount of fisetin was calculated using a fluorescence calibration curve.

The data of fisetin concentration in the flow at various time intervals were collected in the following Table 4.

TABLE 4
Concentration values of fisetin nanoparticles after incubation
with intestinal epithelial tissue for 5 or 16 hours.

| Concentration of | Concentration of | Concentration of |
|---|---|---|
| fisetin nanoparticles | fisetin nanoparticles | fisetin nanoparticles |
| added to intestinal | that have crossed the | that have crossed the |
| epithelial tissue | epithelium after 5 h | epithelium after 16 h |
| (μM) | (μM) | (μM) |
| 1550 ± 28.28 | 73.44 ± 7.14 | 436.17 ± 7.95 |

As is evident from the data in Table 4, less than 5% of the initial concentration had crossed the epithelium after 5 hours of incubation, but already within 16 hours said fisetin concentration value had reached as much as 30% of the initial value.

The test therefore revealed the effective ability of the nanoparticles according to the invention to cross the intestine.

This data is particularly significant as it demonstrates the ability of nanoparticles to make fisetin stable over time as well as to prevent its degradation.

In terms of pharmacological profile, this results in the possibility of administering a lower dose of active ingredient to obtain the same repeatable biological effect in the context of osteogenic differentiation, thus reducing the risk of the onset of any toxicity problems.

### Example 5—Culture Test for Detecting the Osteogenic Effects of Fisetin Nanoparticles in PLGA

To test the ability of fisetin nanoparticles in PLGA according to the invention to induce osteogenic differentiation, human mesenchymal stem cells (hMSC, PromoCell, Heidelberg, Germany), cultured at a density of 5×10⁴ cells with mesenchymal stem cell growth medium (PromoCell) and incubated at 37° C. in a humidified atmosphere with 5% CO₂, were cultured in the presence of fisetin nanoparticles in PLGA further containing fluorescein isothiocyanate (CAS 27072).

In particular, the fisetin nanoparticles in PLGA further containing fluorescein isothiocyanate were prepared by dissolving 20 mg of PLGA in a mixture of 640 μL of acetone and 360 μL of a solution of fisetin/fluorescein isothiocyanate in DMSO having a 1:1 molar ratio between fisetin and fluorescein isothiocyanate, and then applying the same method of the invention described in Example 1.

The analyzes carried out using mesenchymal stem cells in osteogenic differentiation cultured in the presence of fisetin in PLGA conjugated with fluorescein isothiocyanate (to trace the diffusion) showed that after 4 hours of treatment with fisetin nanoparticles in PLGA further containing fluorescein isothiocyanate, fisetin began to spread into the intercellular spaces. After only 6 hours of treatment, fisetin was already clearly visible inside the cells by which it had been completely absorbed.

The experiment was also carried out in parallel with free fisetin not retained within PLGA nanoparticles, observing in particular the value trends for the osteogenic transcription factors RUNX2 and SP7, as well as for the protein COL1A1, after 7 days of incubation.

The data obtained for the two osteogenic factors are shown in the following Table 5.

TABLE 5
Values of the two osteogenic transcription factors RUNX2 and
SP7 in case of treatment of MSC cells with free fisetin or with
fisetin nanoparticles in PLGA according to the invention.

| | RUNX2 (amplification | SP7 (amplification |
|---|---|---|
| | of RUNX2 expression | of SP7 expression |
| | in the sample vs | in the sample vs |
| MSC treated with: | control) | control) |
| Fisetin nanoparticles in | 4.05 | 2.88 |
| PLGA according to the | | |
| invention | | |
| Free fisetin | 2.7 | 1.8 |
| Control (untreated cells) | 1.0 | 1.0 |

As is evident from the results shown in Table 5, the administration of fisetin nanoparticles in PLGA according to the invention was even more effective in stimulating the osteogenic differentiation of the cells than the direct administration of fisetin alone.

The following Table 6 shows the values of the ratios between the optical density due to the presence of COL1A1 and the optical density due to the presence of beta actin protein, normally present in cells and used as an internal control, measured in the two samples.

TABLE 6
COL1A1 protein values after 7 days of incubation
with fisetin nanoparticles in PLGA according
to the invention or with the control.

| | COL1A1 (ratio between COL1A1 |
|---|---|
| | optical density and beta actin |
| MSC treated with: | protein optical density) |
| Fisetin nanoparticles in PLGA | 2.2 |
| according to the invention | |
| Control (free fisetin) | 1.2 |

The data obtained for the value of the protein COL1A1 in case of administration of the fisetin nanoparticles in PLGA according to the invention, compared to the value obtained in case of the control sample, in which free fisetin was used, were consistent with the trends of the osteogenic differentiation factors, as they showed a clearly higher value of the protein COL1A1 in the case of incubation with the fisetin nanoparticles in PLGA according to the invention.

### Example 6—Culture Test for Detecting the Osteogenic Effects of Fisetin on Pediatric Patients Exhibiting RUNX2 Mutations

The positive results obtained using the fisetin nanoparticles in PLGA according to the invention on MSC cell cultures, and in particular the evidence of a significant increase in the osteogenic factor RUNX2, led the inventors to verify the effect of administering fisetin to cells affected by severe decompensation of RUNX2, such as those extracted from pediatric patients affected by cleidocranial dysplasia (CCD), i.e. cells typically characterized by RUNX2 mutations.

Fibroblast-like cells were therefore collected for the experiment from 2 patients carrying mutations in exon 7 of a RUNX2 allele (P1 and P2) and from a healthy patient (ND).

Experiments were therefore carried out, for each type of cell sample, determining the values of RUNX2 obtained in the absence of fisetin (control) or in the presence of fisetin. The amplification of the RUNX2 measured value in the presence of fisetin compared to the control, in the three cases, is shown in the following Table7.

TABLE 7
RUNX2 amplification values in cell samples from
healthy donor (ND) and from 2 patients (P1
and P2) whether or not treated with fisetin.

| | RUNX2 (amplification of RUNX2 |
|---|---|
| | expression in the fisetin- |
| | treated sample compared to the |
| Sample | same untreated sample) |
| Cells from healthy donor (ND) | 1.48 |
| Cells from patient affected by | 1.1 |
| cleidocranial dysplasia (P1) | |
| Cells from patient affected by | 1.08 |
| cleidocranial dysplasia (P2) | |

As is evident from the data shown in Table 7, while in the cells deriving from the healthy donor (ND) the administration of fisetin performed an important action, evidenced by an increase in RUNX2 of about 50% and correlated to an increase in osteogenic induction, i.e. the number of cells that will become osteoblasts, completely in agreement with the evidence of the experiments described in the previous Example 5, in the cells deriving from the two ill patients (P1 and P2) RUNX2 values similar to those of the untreated cells (ratio of amplification which in fact turned out to be about 1, i.e. equal to the theoretical value attributable to no effect) were obtained, thus highlighting that fisetin was unable to produce any effects on the transcription factor RUNX2 in the two ill patients.

This is clearly attributable to the mutation of the RUNX2 transcription factor in patients affected by CCD which is not modulated by treatment with fisetin; being RUNX2 involved in the induction of osteogenic differentiation, fisetin therefore fails to increase the number of cells that can become osteoblasts in patients.

In the same three samples (ND, P1 and P2) the amplification level of the SPARC gene was also measured (in case of absence or presence of fisetin); the results are shown in the following Table 8.

TABLE 8
SPARC amplification values in cell samples from
healthy donor (ND) and from 2 patients (P1
and P2) whether or not treated with fisetin.

| | SPARC (amplification of SPARC |
|---|---|
| | expression in the fisetin- |
| | treated sample compared to the |
| Sample | same untreated sample) |
| Cells from healthy donor (ND) | 2.50 |
| Cells from a patient affected | 2.70 |
| by cleidocranial dysplasia (P1) | |
| Cells from a patient affected | 1.78 |
| by cleidocranial dysplasia (P2) | |

When the cells were cultured in the presence of fisetin, an upregulation of SPARC was observed in all the samples tested, therefore both in the cell sample from the healthy donor (ND), and in the cell samples from the two ill patients (P1 and P2).

The test therefore demonstrated the extraordinary ability of fisetin to restore osteogenic maturation.

This data confirms that fisetin is capable of making the osteoblastic component mature in patient cells, thus increasing the cell production activity and reducing the damage due to RUNX2 mutation.

From the results observed in the experiments, it was therefore possible to conclude that, despite the lack of increase in the osteogenic factor RUNX2 in patients with CCD related to an induction of osteogenic differentiation, the marked and surprising increase in the expression of SPARC, a gene associated with osteogenic maturation, confirmed that fisetin was able to make the osteoblastic component mature, thus increasing the cell production activity and reducing the damage due to the RUNX2 mutation typical of CCD, thus making fisetin a validly usable molecule in the treatment of cleidocranial dysplasia.

Ultimately the experiments showed the particular efficacy, stability, and therefore usability of fisetin nanoparticles in PLGA having a diameter in the range from 120 to 160 nm for making compositions endowed with suitable properties for pharmaceutical use or for use as nutritional supplements.

These particle sizes and properties were obtained thanks to the specific process according to the invention which made it possible, compared to the known processes of the prior art, to operate with a much simpler method, with a single emulsion instead of a double one, as well as, an even more important aspect, to provide fisetin not complexed with other molecules, capable of interacting directly with PLGA and therefore of being then released in a free, immediately bioavailable form.

Furthermore, the fisetin nanoparticles in PLGA according to the invention revealed surprising effects on osteogenic differentiation and on the percentage of intestinal filtration, even highlighting an increase in osteogenic differentiation in MSCs, at the same concentration of fisetin, when fisetin was in the form of nanoparticles in PLGA compared to when it was present in a free form.

Therefore, the fisetin nanoparticles in PLGA according to the invention may be effectively used in the pharmaceutical field to promote bone formation and therefore as a method for the prevention and treatment of bone diseases, such as for example osteoporosis.

Likewise, thanks to their activity, they can also be effectively used in the veterinary field, for the treatment of bone diseases in animals.

Furthermore, the use of fisetin nanoparticles in PLGA can be particularly useful in clinical situations characterized by a picture of non-marked bone demineralization, i.e. osteopenia, and therefore not yet susceptible to pharmacological treatment, or after completion of a specific therapy for the primary or secondary prevention of skeletal fractures, for example in cases of interruption of treatment with antiresorptive or anabolic steroids, where it could be effectively associated with the administration of vitamin D, or still in association with common antiresorptive therapies to promote osteoblastic activity and optimize the treatment. Likewise, the use of fisetin nanoparticles in PLGA is indicated in conditions that require acute or chronic treatment with glucocorticoids to prevent the negative effect of the treatment on osteoblast activity.

Finally, the ability of fisetin to stimulate osteoblastic maturation in patients affected by cleidocranial dysplasia suggests the use of the fisetin nanoparticles in PLGA according to the invention also for the treatment of cleidocranial dysplasia.

1. A plurality of fisetin nanoparticles in poly(lactic-co-glycolic) acid (PLGA), wherein each particle has a diameter in the range from 120 to 160 nm.

2. A method of preparing the plurality of fisetin nanoparticles in PLGA according to claim 1, comprising the following steps:
(a) dissolving PLGA and fisetin, in a weight ratio in the range from 15:1 to 20:1, in DMSO, until a clear and homogeneous organic phase is obtained;
(b) adding dropwise and under stirring said organic phase obtained in step (a) to an aqueous solution of PVA and then allowing the organic phase to evaporate slowly, thus obtaining an aqueous suspension;
(c) centrifuging the aqueous suspension of step (b) and eliminating the supernatant in order to obtain the fisetin nanoparticles in PLGA.

3. The method according to claim 2, wherein the DMSO of step (a) is 100% (w/w) DMSO.

4. The method according to claim 2, wherein the PVA used in step (b) is an aqueous solution of PVA at a concentration from 0.3 to 1%.

5. The method according to claim 2, wherein the centrifugation of step (c) is carried out at a temperature comprised between 0 and 10° C.

6. A plurality of the fisetin nanoparticles in PLGA according to claim 1, wherein said plurality of nanoparticles has a polydispersity index PDI in the range from 0.09 to 0.13.

7. An aqueous suspension of the plurality of fisetin nanoparticles in PLGA according to claim 1, wherein the solution is a phosphate buffered saline (PBS) at pH 7.4 or an aqueous solution of sodium chloride at a concentration of about 0.9% (w/w).

8. A composition comprising the plurality of nanoparticles according to claim 1 and at least one excipient.

9. A nutraceutical composition comprising the plurality of particles according to claim 1 and nutraceutically acceptable excipients.

10.-11. (canceled)

12. The composition according to claim 8, wherein fisetin is present in a unit dosage comprised between 50 and 110 mg.

13. The composition according to claim 9, wherein said composition is an oral composition.

24. 24 (canceled)

25. The plurality of fisetin nanoparticles in PLGA according to claim 1, wherein each particle has a diameter in the range from 130 to 150 nm.

26. The plurality of fisetin nanoparticles in PLGA according to claim 1, wherein each particle has a diameter in the range from 135 to 143 nm.

27. A composition comprising the aqueous suspension of the plurality of particles according to claim 7 and at least one excipient.

28. A nutraceutical composition comprising the aqueous suspension of the plurality of particles according to claim 7 and nutraceutically acceptable excipients.

29-30. (canceled)

31. The composition according to claim 13, wherein the oral composition is in the in the form of granules, powder, syrup, tablet, capsule, gel, aqueous suspension or liquid.

32. A method for the prevention and/or treatment of a bone disease comprising the step of administering the composition according to claim 8.

33. The method according to claim 32, wherein the bone disease is osteopenia, osteoporosis or cleidocranial dysplasia and/or the prevention is the primary or secondary prevention of skeletal fractures.

34. The method for according to claim 32, wherein the composition is administered in association with antiresorptive therapy, glucocorticoid therapy or vitamin D.

38-38. (canceled)

39. A method for the prevention and/or treatment of a bone disease comprising administering the composition according to claim 27.

40. The method according to claim 39, wherein the bone disease is osteopenia, osteoporosis or cleidocranial dysplasia and/or the prevention is the primary or secondary prevention of skeletal fractures.

41. The method according to claim 39, wherein the composition is administered in association with antiresorptive therapy, glucocorticoid therapy and/or vitamin D.

42.-43. (canceled)

44. The composition according to claim 27, wherein said compositions is a veterinary composition.

45. (canceled)

46. The composition according to claim 8, wherein said composition is a veterinary composition.
